(19) United States
(12) Patent Application Publication
(10) Pub. No.: US 2010/0280499 A1
(43) Pub. Date: Nov. 4, 2010

(54) SYSTEM AND METHOD FOR GLYCEMIC CONTROL

(76) Inventors: Ofer Yodfat, Maccabim-Reut (IL); Gali Shapira, Haifa (IL)

Correspondence Address: MINTZ LEVIN COHN FERRIS GLOVSKY & POPEO, ONE FINANCIAL CENTER, BOSTON, MA, 02111, US

(21) Appl. No.: 12/810,634

(22) PCT Filed: Dec. 25, 2008
(86) PCT No.: PCT/IL08/01668

§ 371 Date: Jul. 6, 2010

Related U.S. Application Data
(60) Provisional application No. 61/009,296, filed on Dec. 26, 2007.

(51) Int. Cl. A61M 5/14

(52) U.S. Cl. 604/890.1

(57) ABSTRACT

Disclosed is a glucose monitoring system. The system includes a glucose sensor to periodically perform a plurality of glucose measurements in interstitial fluid, and a processor to determine one or more HbA1c values representative of a patient's glycosylated hemoglobin levels based on the periodic glucose measurements. In some embodiments, the glucose sensor is coupled to a therapeutic fluid (e.g., insulin) dispensing pump.

### CROSS-REFERENCE TO RELATED APPLICATIONS

This application claims priority to provisional U.S. application Ser. No. 61/009,296 entitled “DEVICE AND METHOD FOR GLYCEMIC CONTROL”, and filed Dec. 26, 2007, the content of which is hereby incorporated by reference in its entirety.

### FIELD OF THE INVENTION

The present disclosure relates generally to a system, device and method for assessing the glycemic control of diabetes patients. More particularly, the disclosure relates to a system (or device) that continuously and/or periodically, monitors bodily analytes and can continuously and/or periodically deliver therapeutic fluids. The disclosure also relates to a system that contains a glucose sensor and a method for assessing the user glycemic status, and can include a portable insulin dispenser. Additionally, the disclosure relates to a system which includes an insulin dispenser and which can continuously and/or periodically monitor levels of bodily glucose and a method to assess the user's glucose variability and hemoglobin A1C (HbA1c).

### BACKGROUND

### Diabetes and Glycemic Control

Diabetes mellitus is a disease of major global importance, increasing in frequency at almost epidemic rates, such that the worldwide prevalence of the disease in 2006 was 170 million people and is predicted to at least double over the next 10-15 years. Diabetes is characterized by a chronically raised blood glucose concentration (hyperglycemia), due to a relative or absolute lack of the pancreatic hormone, insulin. Within the healthy pancreas, beta cells, located in the islets of Langerhans, continuously produce and secrete insulin according to the blood glucose levels, thus maintaining near constant glucose levels in the body.

Diabetes can cause acute and chronic complications. Acute complications include hypoglycemia and ketoacidosis. Long-term complications, due to the affect on small and large blood vessels, include eye, kidney, and nerve damage and accelerated atherosclerosis, with increased rates of coronary heart disease, peripheral vascular disease and stroke.

The Diabetes Control and Complications Trial (DCCT) demonstrated that development and progression of the chronic complications of diabetes are greatly related to the degree of altered glycemia as quantified by determinations of glyco Hemoglobin (HbA1c). [DCCT Trial, N Engl J Med 1993; 329: 977-986, UKPDS Trial, Lancet 1998; 352: 837-853. BMJ 1998; 317, (7160): 703-13 and the EDIC Trial, N Engl J Med 2005; 353, (25): 2643-53].

### Insulin Infusion Pumps

Frequent insulin administration can be done by multiple daily injections (MDI) with syringe or by continuous/periodic subcutaneous insulin injection (CSII) performed by insulin pumps. In recent years, ambulatory portable insulin infusion pumps have emerged as a superior alternative to multiple daily injections of insulin. These pumps, which deliver insulin at a continuous/periodic basal rate as well as in bolus volumes, were developed to liberate patients from repeated self-administered injections, and to enable greater flexibility in dose administration.

Several ambulatory insulin infusion devices are currently available on the market. The first generation of such devices employs disposable syringe-type reservoir and tubes. These devices have been described, for example, in U.S. Pat. Nos. 3,771,694, 4,657,486 and 4,498,843, the contents of all of which are hereby incorporated by reference in their entireties. A drawback of these devices is their large size and weight, caused by their spatial configuration and the relatively large driving mechanism associated with the syringe and the piston. These relatively bulky devices have to be carried in a patient's pocket or be attached to a belt. Consequently, the fluid delivery tube becomes long, e.g., longer than 40 cm, to enable needle insertion in remote locations of the body. These uncomfortable bulky devices with a long tube are disfavored by many diabetic insulin users because they disturb regular activities, such as sleeping, physical activities (e.g., swimming), etc. In addition, the long delivery tube is not suitable for use with some optional remote insertion sites such as the buttocks and the extremities.

To avoid the shortcomings associated with the necessity of using long delivery tube, a new concept, implemented as second generation devices, was proposed and developed.

Devices and systems based on the second generation concept included a housing having a bottom surface adapted for contact with the patient's skin, a reservoir disposed within the housing, and an injection needle adapted for communication with the reservoir. These skin adherable devices could be disposed of every 2-3 days, as is the case with current pump infusion sets. These devices, conforming to the second generation paradigm, are described, for example, by Schneider in U.S. Pat. No. 4,498,843, Burton in U.S. Pat. No. 5,957,895, Connelly, in U.S. Pat. No. 6,589,229, and by Flaherty in U.S. Pat. No. 6,740,059, the contents of all of which are hereby incorporated by reference in their entireties. Other configurations of skin securable (e.g., adherable) pumps are disclosed, for example, in U.S. Pat. Nos. 6,723,072 and 6,485,461, the contents of all of which are hereby incorporated by reference in their entireties. In these patents, the pump is generally configured as a single piece securable (adherable) to the patient skin for the entire usage duration. The needle emerges from the bottom surface of the device and is fixed to the device housing. A disadvantage of these second-generation skin securable (e.g., adherable) devices lies in the fact that they are generally expensive, bulky and heavy.

To avoid these shortcomings, a 3^{rd} generation of skin securable (e.g., adherable) pump devices were proposed and developed, as described, for example, in U.S. patent application Ser. No. 11/397,115, assigned to Medingo, the content of which is hereby incorporated by reference in its entirety. This pump is configured as a miniature portable programmable dispensing patch that has no tubing and that can be attached to the patient's skin. In some embodiment, this pump includes two parts, a disposable part that contains a reservoir and an outlet port, and a reusable part that contains the electronic parts, a driving mechanism, and other types of relatively expensive components and/or units. This pump device may, in some embodiments, include a remote control unit that enables data acquisition, programming, and entry of user inputs.

### Insulin Pump and Continuous Glucose Monitors (CGM)

Insulin pumps can communicate with analyte sensors, such as a continuous glucose monitor (CGM), as described for example in U.S. Pat. No. 6,558,351, the content of which is hereby incorporated by reference in its entirety. These discrete devices (i.e., the sensor and the pumping device) are relatively bulky, expensive' devices that require two infusion sets with long tubing and two insertion sites. A new generation of a dual function device is described, for example, in U.S. patent applications Nos. 11/706,606 and 11/963,481, and International Patent Application No. PCT/IL07/001,579, assigned to Medingo Ltd., the contents of which are hereby incorporated by reference in their entireties. That device is a single skin securable (adherable) patch employing a single subcutaneous cannula configured to perform drug infusion and analyte sensing.

### Hemoglobin A1c

Glycemic control is a medical term referring to disease severity status in a person with diabetes mellitus, or the ability to maintain normal glucose levels and to avoid complications (neuropathy, nephropathy, retinopathy, etc.).

Glycosylated hemoglobin (HbA1c) is a form of hemoglobin used primarily to identify the patient glycemic control over prolonged periods of time (e.g., 3 months). It is formed in a non-enzymatic pathway by hemoglobin's normal exposure to high plasma levels of glucose. A high HbA1c represents poor glycemic control.

The approximate mapping between HbA1c values and average blood glucose measurements over the previous 4-12 weeks is shown in the table depicted in FIG. 1 (table available from public sources).

The above-referenced table is inaccurate, partly because the level of HbA1c does not reflect the weighted mean of the preceding measured bodily glucose concentration, but rather a simple mean. BG excursions that are more recent generally contribute more than earlier events to a currently measured HbA1c level.

Currently, diabetes patients may monitor the glycemic status by periodic measurements of their HbA1c levels. A follow-up visit is usually required to obtain and discuss the results. Recently, mathematical models have been developed that exhibit the relationship between HbA1c and blood glucose. One such model is the model developed by the team of Siv M. Ostermann-Golker and Hubert W. Vesper (Journal of Diabetes and its Complications 2006, 20,285-294).

### SUMMARY OF THE EMBODIMENTS

In some embodiments, a system that can periodically assess the HbA1c and/or glucose variability of a user by frequent bodily glucose measurements is provided.

In some embodiments, a system that can continuously monitor glucose levels configured to assess the user's HbA1c value and/or glucose variability is provided.

In some embodiments, a system/device that includes a skin adherable continuous glucose sensing patch configured to assess the user's HbA1c value and/or glucose variability is provided.

In some embodiments, an insulin dispensing and glucose sensing system configured to assess the user's HbA1c value and/or glucose variability is provided.

In some embodiments, an insulin dispensing and continuous glucose sensing system configured to assess the user's HbA1c value and/or glucose variability is provided.

In some embodiments, a system which is miniature, discreet, economical for the users and cost effective that is configured to assess the user's HbA1c value and/or glucose variability is provided.

In some embodiments, a system that includes a continuous glucose sensing patch that can be remotely controlled and that is configured to assess the user's HbA1c value and/or glucose variability is provided.

In some embodiments, a system that includes a continuous glucose sensing and insulin dispensing patch that can be remotely controlled and that is configured to assess the user's HbA1c value and/or glucose variability is provided.

In some embodiments, a system that includes a miniature skin adherable glucose sensing patch that can continuously and/or periodically dispense insulin and monitor body glucose concentration levels, and that is configured to assess the user's HbA1c value and/or glucose variability is provided.

In some embodiments, a system that includes a miniature skin adherable glucose sensing and insulin dispensing patch that can continuously/periodically dispense insulin and monitor body glucose concentration levels, and that is configured to assess the user's HbA1c value and/or glucose variability is provided.

The present disclosure describes a system that continuously monitors bodily analyte levels (e.g., glucose) and can deliver therapeutic fluid into the body (e.g., insulin).

In some embodiments, the system includes a continuous glucose monitoring unit (CGM) that can periodically assess the user's glycemic control by providing a HbA1c value that is derived from a mathematical model that exhibits the relationship between HbA1c values and bodily glucose concentrations. The user's glycemic control may be determined at any given time, based on the glycemic control during the preceding 120 days (e.g., based on BG measurements over the last 120 days, from which HbA1c values may be determined). The user's glycemic control can also be determined (e.g., computed) according to the user's glucose variability. In some embodiments, the system comprises, in addition to a CGM, an insulin dispensing unit. The CGM and insulin dispensing unit may be disposed in a single housing. In some embodiments, a remote control unit may be used that is configured to communicate with the CGM and dispensing patch unit and to enable programming of therapeutic fluid delivery, user input and data acquisition. In some embodiments, the CGM and dispensing patch unit arrangement is composed of two parts—a disposable part and reusable part. The disposable part contains a reservoir and outlet port. The reusable part contains, in some embodiments, electronics (PCB, processor, etc), a driving mechanism a metering portion, and other relatively expensive components. In some embodiments, a glucometer may be integrated into the remote control unit to enable calibration of the CGM (i.e., a calibration unit). In some embodiments, the fluid delivered by the sensing and dispensing patch unit is adjusted by a processor according to the detected glucose concentrations in a closed or semi-closed loop system.

In some embodiments, a user's HbA1c levels may be determined based on a mathematical model representing the relationship between HbA1c values and bodily glucose concentrations.

In some embodiments described herein, a system that can periodically determine the HbA1c and/or glucose variability of the user by frequent bodily glucose measurements is provided.

In some embodiments described herein, a system for continuous/periodic glucose sensing configured to assess the user's HbA1c value and/or glucose variability is provided.

In some embodiments described herein, a system that comprises a skin adherable continuous glucose sensing patch configured to assess the user's HbA1c value and/or glucose variability is provided.

In some embodiments described herein, a system that can continuously/periodically dispense insulin and monitor glucose levels and configured to assess the user's HbA1c value and/or glucose variability is provided.

In some embodiments described herein, a system which comprises a glucose sensing and insulin dispensing unit that is miniature, discreet, economical for the users and cost effective, and that is configured to assess the user's HbA1c value and/or glucose variability, is provided.

In some embodiments described herein, a system that comprises a continuous glucose sensing patch that can be remotely controlled and that is configured to assess the user's HbA1c value and/or glucose variability is provided.

In some embodiments described herein, a system that comprises a continuous glucose sensing and insulin dispensing patch that can be remotely controlled and that is configured to assess the user's HbA1c value and/or glucose variability is provided.

In some embodiments described herein, a system that comprises a miniature skin adherable glucose sensing patch that can continuously and/or periodically dispense insulin and monitor body glucose concentration levels and that is configured to assess the user's HbA1c value and/or glucose variability is provided.

In some embodiments described herein, a system that comprises a miniature skin adherable glucose sensing and insulin dispensing patch unit that can continuously dispense insulin and monitor body glucose concentration levels and that is configured to assess the user's HbA1c value and/or glucose variability is provided.

In some embodiments, the HbA1c assessment is established via processing of information obtained from frequent glucose measurements (e.g., subcutaneous continuous glucose monitoring).

In some embodiments, the HbA1c assessment can be obtained from a mathematical model exhibiting the relationship between HbA1c and interstitial fluid (ISF) glucose.

In some embodiments, the HbA1c assessment can be obtained from two mathematical models: 1) exhibiting the relationship between HbA1c and blood glucose, and 2) exhibiting the relationship between ISF and blood glucose.

In some embodiments, a mathematical model exhibiting the relationship between HbA1c and blood glucose can be applied to ISF glucose avoiding the need for a model that transforms ISF glucose to BG.

In some embodiments, the HbA1c can be assessed using one or more of conventional models, e.g., the mathematical model exhibiting the relationship between HbA1c level and blood glucose developed by the team of Siv M. Ostermann-Golker and Hubert W. Vesper (Journal of Diabetes and its Complications 2006, 20,285-294). Alternatively, simpler models may be applied, for example, the model developed by Beach (1979) which is based on simple first order reaction kinetics between glucose and hemoglobin, loss of HbA1c through erythrocyte clearance, and in which HbA1c is assumed to being a stable reaction product.

In some embodiments, the applied model requires inputting the time-weighted averaged bodily glucose values over a predefined period, e.g., 1 day. That is, the level of HbA1c does not reflect the simple mean, but rather a weighted mean of the preceding measured bodily glucose concentration. More recent BG events may thus contribute relatively more to the final HbA1c result than earlier events.

In some embodiments, 50% of HbA1c assessed value is determined by the BG levels during the preceding 35 days, 25% by the BG levels during 25 day period before that period, and the remaining 25% during the 2 month period before these periods (as indicated, for example, in Diabetes Care, 2006, 29, (2), 466-467).

In some embodiments, if a high HbA1c value is assessed, the user gets an alert from the system recommending a medical check by a health practitioner (e.g., to perform a dilated eye examination, serum creatinine, etc.), and possibly performing pump setting changes.

In some embodiments, glucose variability may be determined using a continuous glucose sensor, and may be considered alone, or in combination with the assessed HbA1c value as an indicator of glycemic control and predictor of long-term complications (e.g., retinopathy, nephropathy).

In some embodiments, glucose variability is defined by one or more of, for example, standard deviation of bodily glucose values, duration of normal, low or high readings, mean amplitude of glycemic excursions and/or glycemic lability index (as indicated, for example, in Diabetes 2004, 53, 955-962).

In some embodiments, the HbA1c value assessment procedure is implemented in a system that continuously and/or periodically monitors body glucose levels. Such a system may be a patch unit that can be adhered to the user's skin. The patch unit may comprise a reusable part and a disposable part. In some embodiments, the system comprises, in addition to the CGM patch unit, a remote control unit wherein a blood glucose monitor (e.g., glucometer) is integrated in the remote control unit for periodic calibration of the CGM patch unit.

The blood glucose monitor (e.g., glucometer) may alternatively be integrated in the reusable part of the CGM patch unit of the system.

The HbA1c value assessment procedure may be implemented in the remote control unit of the system. Alternatively, the HbA1c value assessment method could be implemented in the reusable part of the CGM patch unit of the system.

In some embodiments, the HbA1c value assessment procedure is implemented in a system that continuously monitors body glucose levels and can concomitantly deliver insulin into the body. The system may be a patch unit that can be secured (e.g., adhered) to the user's skin. The patch unit may include a reusable part and a disposable part. The insulin dispensing and glucose sensing capabilities can be combined into a semi closed loop system, where a processor-controller apparatus regulates the dispensing of basal insulin according to the sensed glucose concentration. According to some embodiments, the system may include a remote control unit to enables programming of therapeutic fluid delivery, user input and data acquisition.

The HbA1c value assessment procedure may be implemented in the remote control unit of the system. Alternatively, the procedure may be implemented in the reusable part of the CGM and insulin dispensing patch unit of the system. Alternatively, the procedure could be implemented in both the reusable part of the patch unit of the system and the remote control unit of the system.

In one aspect, a glucose monitoring system is disclosed. The system includes a glucose sensor to periodically perform a plurality of glucose measurements in interstitial fluid, and a processor to determine one or more HbA1c values representative of a patient's glycosylated hemoglobin levels based on the periodic glucose measurements.

Embodiments of the system may include one or more of the following features.

The glucose sensor may be coupled to a fluid dispensing pump.

In another aspect, a portable insulin dispensing system is disclosed. The system includes a portable insulin dispensing pump to deliver insulin to a patient, a glucose sensor to periodically perform a plurality of glucose measurements corresponding to glucose concentration in the blood, and a processor to determine one or more HbA1c values representative of a patient's glycosylated hemoglobin levels based on the periodic glucose measurements.

Embodiments of the system may include any of the features described in relation to the first system above, as well as any of the following features.

The processor may be provided in any one or more of, for example, the portable insulin dispensing system, a handheld remote control unit for the insulin dispensing pump and/or a handheld blood glucose monitor.

The glucose sensor may be configured to periodically perform a plurality of glucose measurements in interstitial fluid.

The glucose sensor may be coupled to the dispensing pump.

The processor may be further configured to compute one or more values representative of the patient's glucose variability levels.

At least part of the pump may be securable to the patient's skin.

The pump may include the glucose sensor.

The pump may include a disposable part and a reusable part.

The one or more HbA1c values may be determined based on a mathematical model relating the one or more HbA1c values to glucose concentrations determined from the glucose measurements. The mathematical model may include a first order reaction kinetics model representative of a first order reaction kinetics between glucose and hemoglobin and loss of HbA1c through erythrocyte clearance in which HbA1c is estimated to be a stable reaction product.

The one or more HbA1c values may be computed based on a time-weighted averaged computation of the glucose measurements.

In a further aspect, a method for monitoring glycemic control in a patient is disclosed. The method includes providing a skin securable housing including a glucose sensor, periodically performing a plurality of glucose measurements in interstitial fluid, and determining one or more HbA1c values representative of a patient's glycosylated hemoglobin levels based on the periodic glucose measurements.

Embodiments of the method may include one or more of the features described in relation to the above systems, as well as any of the following features.

Determining the one or more HbA1c values may include determining the one or more HbA1c values using a processor disposed in any one or more of, for example, the skin securable housing, a handheld remote control unit for an insulin dispensing pump and/or a handheld blood glucose monitor.

Providing the glucose sensor may include providing a glucose sensor coupled to a fluid dispensing pump.

In yet another aspect, a method for monitoring glycemic control in a patient is disclosed. The method includes delivering insulin into the body of a patient using an insulin dispensing device, performing periodically a plurality of glucose measurements, the glucose measurements performed using a sensor coupled to the dispensing device, and determining one or more HbA1c values representative of a patient's glycosylated hemoglobin levels based on the periodic glucose measurements.

Embodiments of the method may include one or more of the features described in relation to the above systems and method, as well as any of the following features.

The method may further include providing a processor in any one or more of, for example, the dispensing device, a handheld remote control unit for the insulin dispensing device and/or a handheld blood glucose monitor, the processor configured to determine the one or more HbA1c values.

Performing periodically the plurality of glucose measurements may include performing a plurality of glucose measurements in interstitial fluid.

The method may further include determining one or more values representative of the patient's glucose variability levels.

Determining the one or more HbA1c values may include computing the one or more HbA1c values based on a mathematical model relating the one or more HbA1c values to the glucose measurements. The mathematical model includes a first order reaction kinetics model representative of a first order reaction kinetics between glucose and hemoglobin and loss of HbA1c through erythrocyte clearance in which HbA1c is estimated to be a stable reaction product.

Computing the one or more HbA1c values may include computing the one or more HbA1c values based on a time-weighted averaged computation of the glucose measurements.

Performing periodically the plurality of glucose measurements may include performing periodically glucose concentration level of the patient using a continuous glucose monitoring unit (CGM).

The method may further include periodically calibrating, using a glucometer, one or more of, for example, the sensor and/or a continuous glucose monitoring unit (CGM) performing the glucose measurements.

### BRIEF DESCRIPTION OF THE DRAWINGS

FIG. 1 is a table of HbA1c and blood glucose averages.

FIGS. 2a-c are schematic diagrams of an exemplary continuous glucose monitoring (CGM) device and a remote control unit configured to perform HbAl c computations.

FIGS. 3a-d are schematic diagrams of an exemplary system that includes a CGM with an insulin dispensing unit and a remote control unit configured to perform HbA1c computations.

FIGS. 4a-b are schematic diagrams of exemplary insulin infusion systems containing two embodiments of continuous subcutaneous glucose monitors to provide blood glucose readings (BG) for the HbA1c computation.

FIG. 5 are equations representative of an exemplary mathematical model developed by the team of Siv M. Ostermann-Golker and Hubert W. Vesper.

FIG. 6 is a block diagram of an exemplary procedure to determine HbA1c levels.

FIG. 7 is an anatomical/physiological schematic depicting an exemplary data acquisition model to determine the glucose concentration levels in the ISF.

FIG. 8 is a diagram of an exemplary embodiment of an HbA1c computation implementation using a remote control unit and PC.

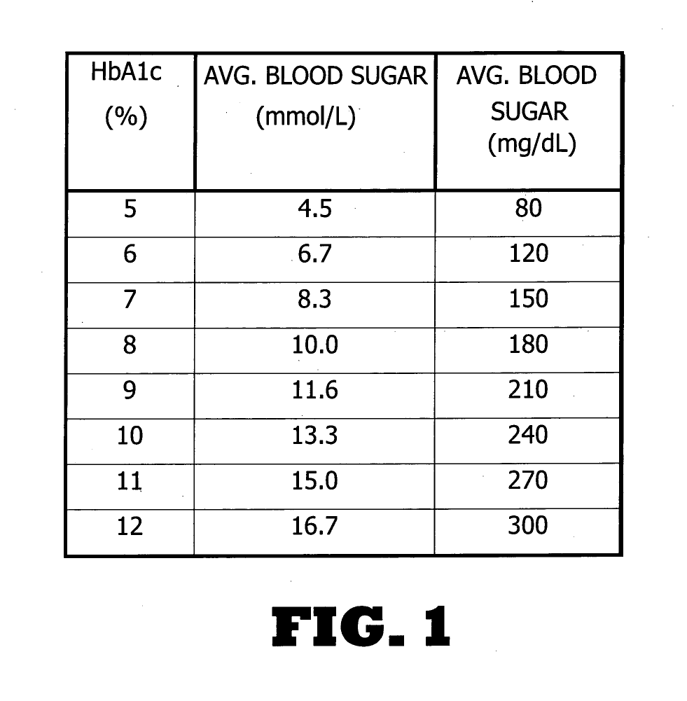

Like reference numbers and designations in the various drawings indicate like elements.

### DETAILED DESCRIPTION

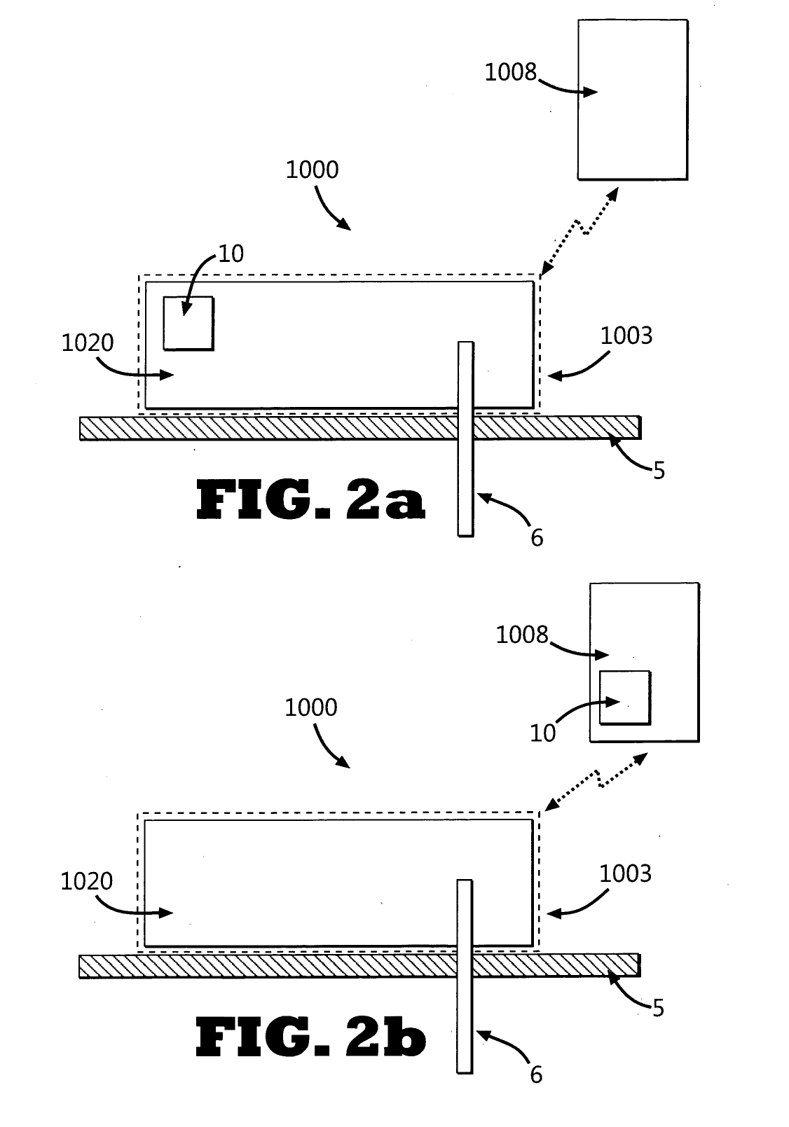

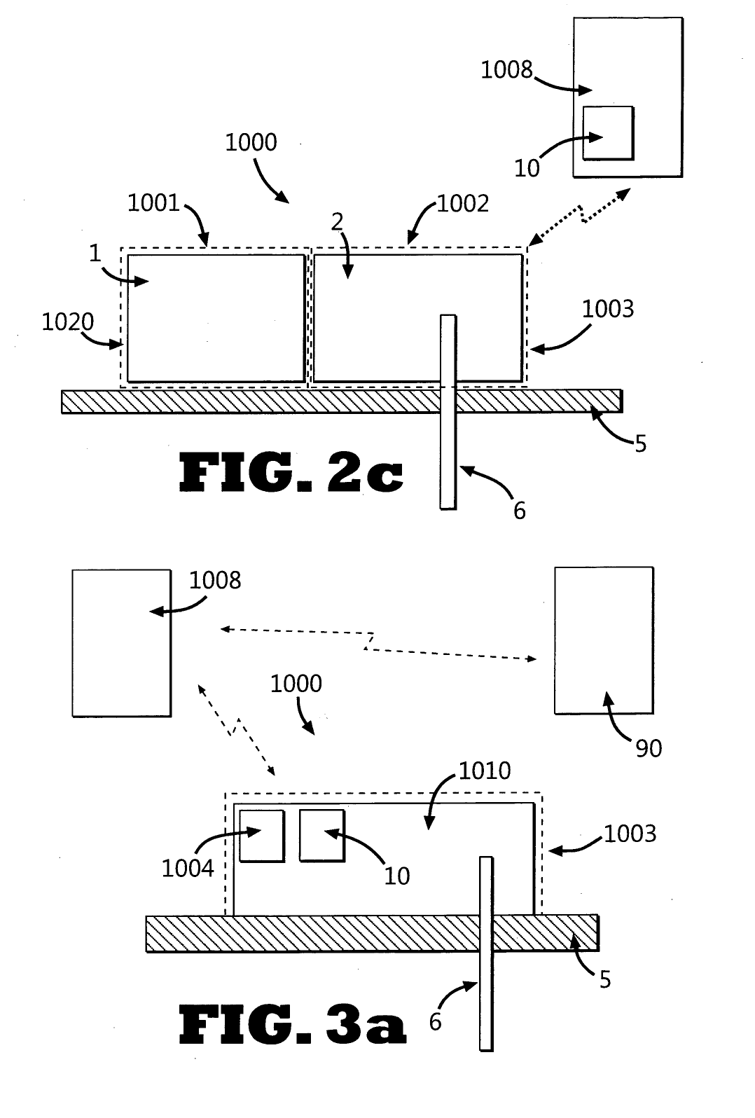

Referring to FIGS. 2a-c, schematic diagrams of exemplary embodiments of a system 1000 for continuous or periodic glucose monitoring (e.g., CGM) are shown. The system 1000 includes, without any limitations, a CGM unit 1020 and optionally a remote control unit 1008. The CGM unit is connected to a cannula 6 that penetrates the patient's skin 5 and resides in the subcutaneous tissue for sensing the glucose concentration in the interstitial fluid (ISF). Other types of probes (e.g., non-tubular probes) to sense and/or measure glucose concentrations in the ISF may be used.

The CGM unit 1020 may be contained in a single housing 1003, as shown, for example, in FIGS. 2a-b, or in two housings 1001, 1002, comprising a reusable part 1 and a disposable part 2 respectively, as shown in FIG. 2c. Data acquisition may be performed by the remote control unit 1008. The CGM unit 1020 may be configured as a patch that can be directly attached to the patient's skin 5 by adhesives (not shown) or, in some embodiments, it may be connected to a dedicated cradle unit (not shown) that is adhered to the patient skin 5 and enables the CGM patch unit 1020 disconnection from and reconnection to the body.

A module to implement HbA1c computation (also referred to as the HbA1c assessment feature) 10 can be located in the CGM unit 1020 (as shown in FIG. 2a), or in the remote control unit 1008 (as shown, for example, in FIGS. 2b-c).

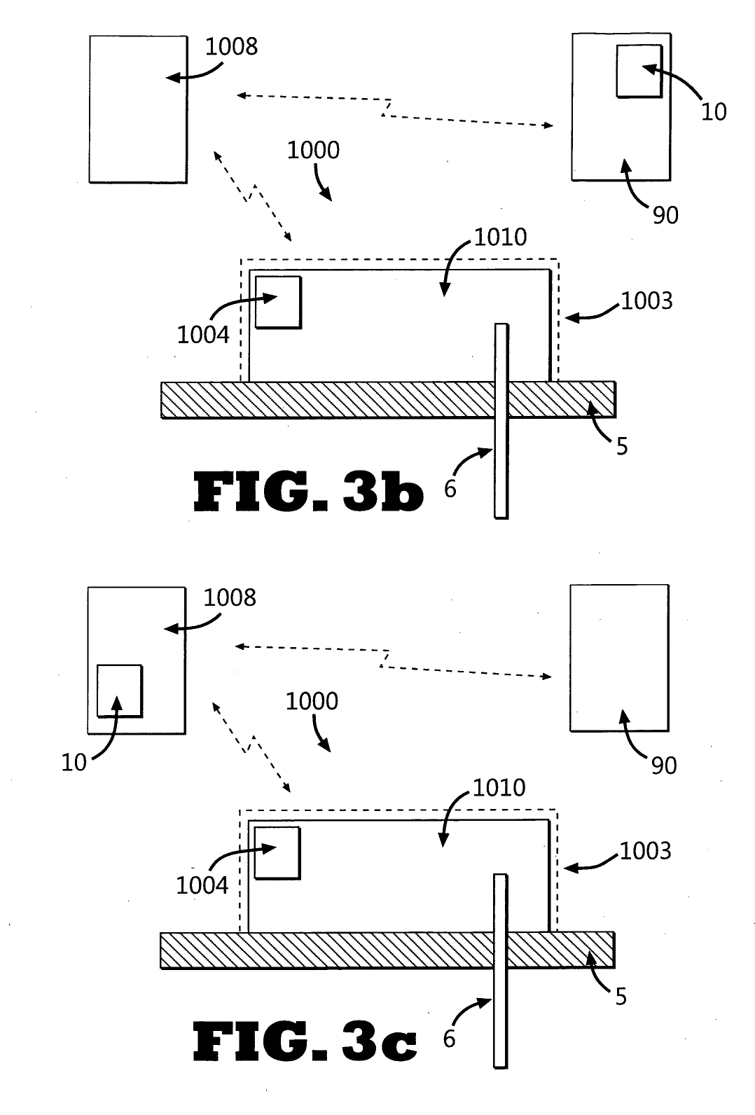

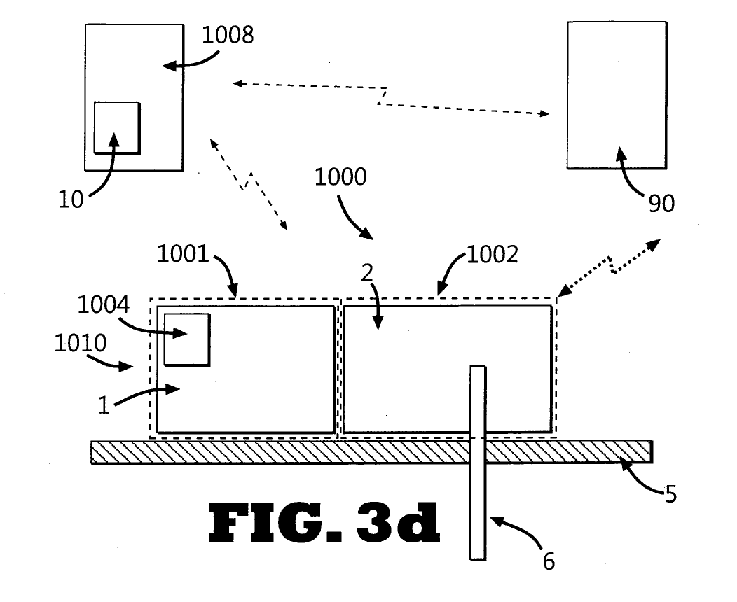

Referring to FIGS. 3a-d, schematic diagrams of exemplary embodiments of a system 1000 configured to perform continuous sensing of ISF glucose levels (CGM) and to dispense therapeutic fluids (e.g., insulin) to the body are shown. The system 1000 includes a sensing and dispensing unit 1010, a remote control unit 1008, and a blood glucose (BG) monitor 90 to enable calibration of the CGM. In some embodiments, the unit 1010 may not include a CGM (for ISF glucose measurements), and thus, under those circumstances, BG measurements performed by a blood glucose monitor 90 may be used for HbA1c determination. The sensing and dispensing unit is connected to a cannula 6 that delivers the drug through the skin 5 into the body. The sensing and dispensing unit can be contained in a single housing 1003, as shown, for example, in FIGS. 3a-c, or in two housings 1001, 1002, comprising a reusable part 1 and a disposable part 2, respectively, as shown, for example, in FIG. 3d. Programming functionality (e.g., to specify infusion/flow profiles) and data acquisition may be performed by a remote control unit 1008, or may alternatively be performed directly by operating buttons 1004 located on the dispensing unit housing. In some embodiments, a BG monitor can be contained within the remote control unit or within the dispensing unit (not shown). The patch unit 1010 as described for example in U.S. Provisional Patent Application Ser. No. 61/123,509, can be directly attached to the patient's skin 5 by adhesives or other types of securing/connection mechanisms (not shown), or can be attached to a dedicated cradle unit (not shown) that is adhered to the patient skin 5 and enables the patch unit 1010 to disconnect from and/or reconnect to the body as described, for example, in co-owned, co-pending International Patent Application No. PCT/IL07/001,578 and U.S. patent application Ser. No. 12/004,837, claiming priority to Provisional Patent Application Ser. No. 60/876,679, the contents of all of which are hereby incorporated by reference in their entireties.

A HbA1c computation implementation 10 may be located in the sensing and dispensing unit 1010 (as shown, for example, in FIG. 3a), the BG monitor 90 (shown, for example, in FIG. 3b) or in the remote control unit 1008 (shown in FIGS. 3c-d).

In some embodiments, the system does not include a glucometer for calibration of the CGM.

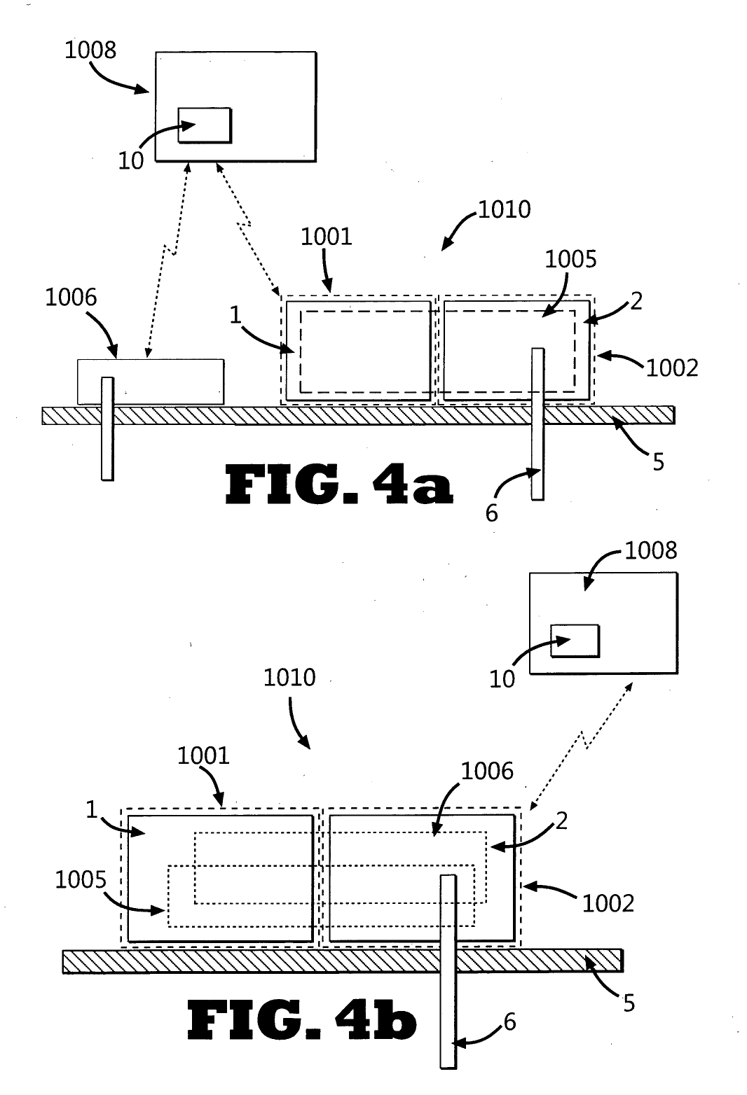

Referring to FIGS. 4a-b, schematic diagrams of an exemplary system 1000 that comprises a two-part patch unit that includes an insulin dispensing patch unit 1010, a remote control unit 1008 and a continuous glucose monitor (CGM) apparatus 1006 are shown. The two part patch unit includes a dispensing apparatus 1005 having a reusable part 1 and a disposable part 2. As shown, the reusable part 1 is contained in a first housing 1001 and the disposable part 2 is contained in a second housing 1002. The HbA1c computation implementation 10 can be contained within the remote control unit 1008 or within the patch unit (not shown). FIG. 4a depicts a stand-alone configuration of the CGM apparatus in which continuous glucose readings can be transmitted to the remote control and patch units (indicated by the arrows). FIG. 4b shows the CGM apparatus 1006 contained within a two part patch unit, which includes a portion contained within the reusable part 1 and a another portion contained within the disposable part 2. The dispensing apparatus 1005 can be connected to a cannula and the CGM apparatus 1006 can be connected to a separate probe (not shown). Alternatively, both apparatuses can be connected to a single cannula/probe as described, for example, in co-owned U.S. application Ser. No. 11/706,606 and 11/963,481, and International Patent Application No. PCT/IL07/001,579, the contents of all of which are hereby incorporated by reference in their entireties. In some embodiments, therapeutic fluid (e.g., insulin) can be dispensed based on, at least in part, CGM readings (i.e., in a closed loop system). In some embodiments, therapeutic fluid can be dispensed according to CGM readings and additional pre-meal bolus inputs (e.g., a semi closed loop system).

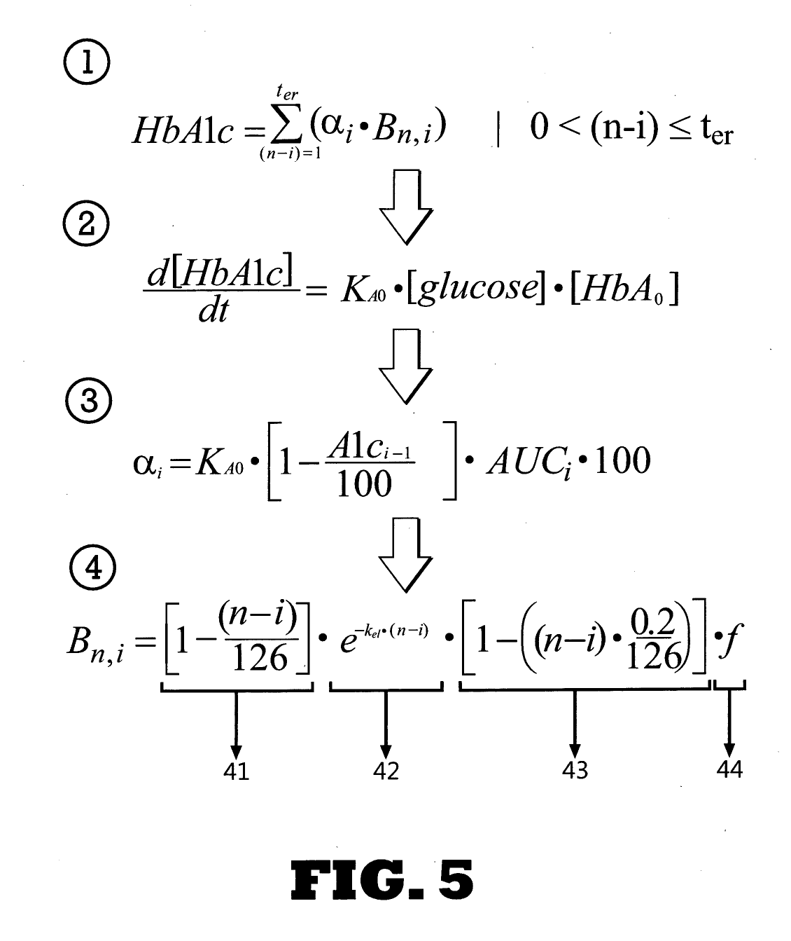

Referring to FIG. 5, an exemplary mathematical representation of the model of the relationship between HbA1c and blood glucose concentrations is shown. The concentration of HbA1c in the blood is determined by the rate of its formation and removal. As described by Siv M. Ostermann-Golker and Hubert W. Vesper (Journal of Diabetes and its Complications 2006, 20,285-294), HbA1c measured at a certain day n can be expressed as the sum of contributions from each of the preceding days i of the 126-day erythrocyte life-span (ter), as represented in the Equation 1, where aᵢ, is the incremental increase in adduct formed at day i and B_{n,i} is a factor that accounts for detraction of this increment. The “adduct” refers to the formation of new HbA1c (which depends primarily on the amount of blood glucose and the amount of unreacted hemoglobin), and the “detraction” refers to a reduction in the level of HbA1c (due, for example, to the erythrocyte turnover and the HbA1c chemical stability).

The rate of formation of HbA1c may be determined in accordance with Equation 2 (shown in FIG. 5), where K_{A0} is the reaction rate. Equation 3 represents the percent ratio of the adduct increment HbA1cᵢ, formed during day i, to HbA1c, denoted as aᵢ. AUCᵢ, is the area under the curve, or dose of glucose, and represents the time-weighted average of glucose concentration over the period of day i.

The removal, or reduction, of HbA1c is expressed as factor B_{n,i}, represented, for example, by Equation 4. The first factor provided in Equation 4, marked as reference numeral 41, accounts for the elimination of HbA1c due to erythrocyte turnover (erythrocyte lifespan-126 days). The second factor of the equation, marked as reference numeral 42, accounts for the loss of HbA1c due to chemical instability, where Kₑₗ is the reaction rate for this type of elimination. The third factor represented in Equation 4, marked as reference numeral 43, accounts for the loss of HbA1c due to spleen facilitated clearance. Spleen facilitated clearance corresponds to an additional path of erythrocytes reduction (or removal). Approximately 20% of the hemoglobin content is lost from the circulating red blood cells due to spleen-facilitated vesiculation, which is most pronounced in old cells. The fourth factor of the equation, noted as 44, accounts for the loss of HbA1c due to other types of elimination. This factor is normally 1, but, under certain circumstances that alter the RBC count (e.g., smoking, high altitude, etc.), the value of this factor may vary.

Based on clinical investigations, examples for the pharmacokinetic parameters K_{A0}, Kₑₗ are given below:

K_{A0} (1 mmol⁻¹h⁻¹)=5*10⁻⁶ (as determined by Higgins and Bunn, 1981)

K_{A0} (1 mmol⁻¹h⁻¹)=7.75*10⁻⁶ (as determined byMortensen and Volund, 1984)

Kₑₗ (d⁻¹)=0.01 (as determined by Bunn et al., 1976)

Kₑₗ(d⁻¹)=0.0045 (as determined by Saunders, 1998)

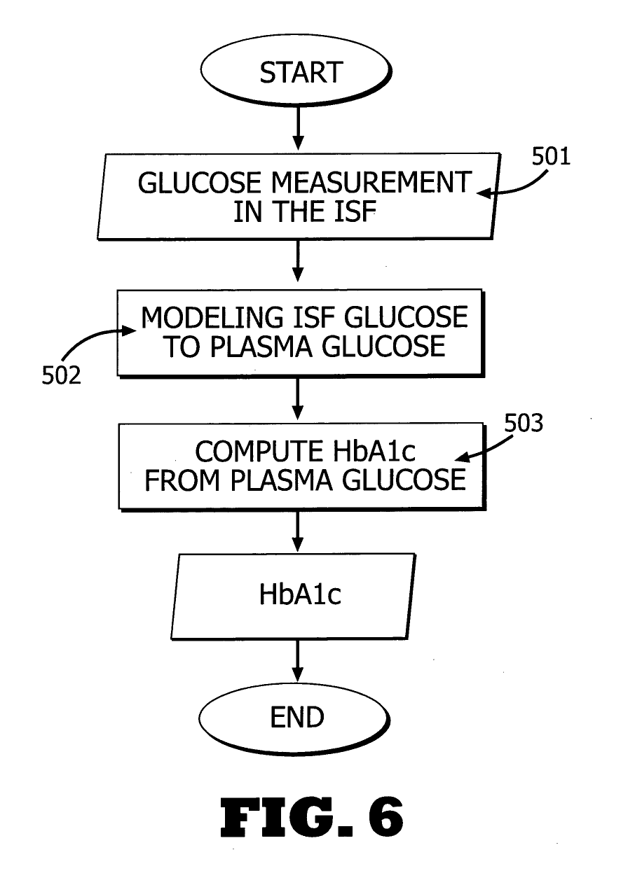

Referring to FIG. 6, a schematic block diagram of an exemplary modeling (or procedural) approach to determine HbA1c level is shown.

Generally, conventional mathematical models representative of the relationship between HbA1c and bodily glucose concentrations require parameters that are determined from blood samples (e.g., plasma glucose). Consequently, a procedure to determine HbA1c levels based on such conventional model requires inconvenient blood sampling. In contrast, the system disclosed herein is attached to the patient's skin and has accessibility to the subcutaneous tissue layer through the cannula or through some other probe (e.g., nom-tubular type probe). Thus, glucose concentration levels in the ISF (“interstitial fluid”) may be determined to thus enable subsequent determination of the HbA1c levels. Accordingly, and as shown in FIG. 6, the glucose concentration levels in the ISF are measured 501.

Having measured the glucose concentration levels, the measured ISF glucose levels are transformed 502 into conventional plasma glucose levels using, for example, a “transitional model”. The modeled plasma glucose is then applied in a model that determines (e.g., computes) 503 the HbA1c from plasma glucose. This “integrated” modeling depicted in FIG. 6 thus enables HbA1c computations using data/parameters which may be acquired using sensing/probing modules or devices disposed, for example, in the adherable patch unit, or disposed in dedicated sensing devices.

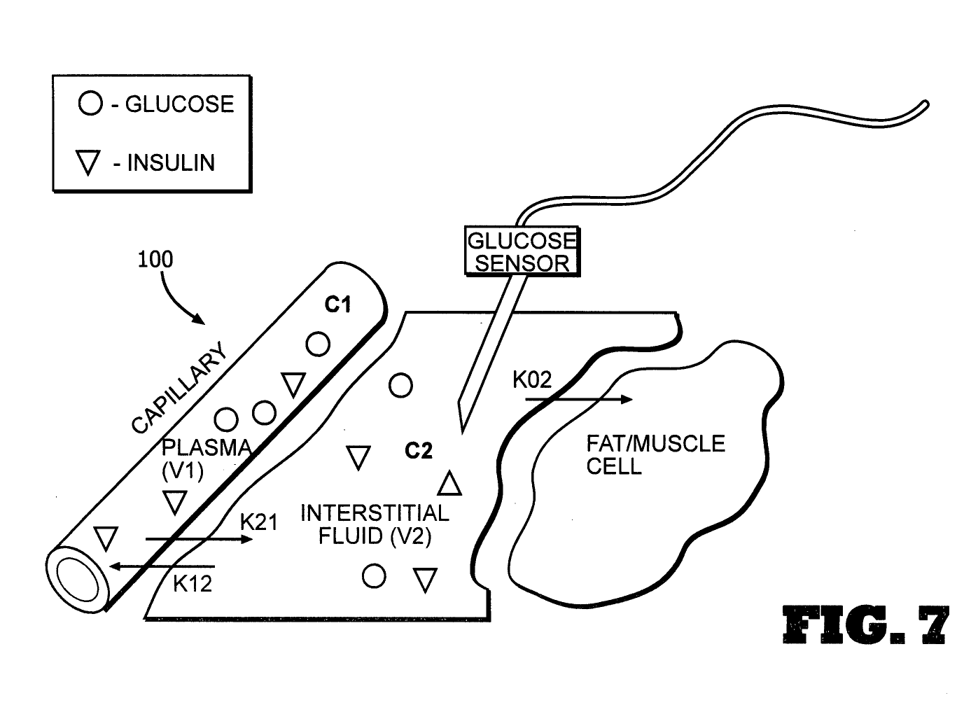

Referring to FIG. 7, an anatomical/physiological schematic depicting an exemplary data acquisition model 100 to determine the glucose concentration levels corresponding to the plasma (based on which the HbA1c levels may be determined) is shown. The exemplary model 100, where subcutaneous glucose is used to estimate plasma glucose, was proposed by K. Rebrin et al. As shown, the model 100 describes plasma (C₁) and interstitial fluid (ISF; C₂) glucose kinetics. In this model it is assumed that ISF glucose equilibrates with plasma glucose by diffusion (D=k₂₁V₁=k₁₂V₂) and that ISF glucose is cleared from ISF by tissue surrounding the sensor (clearance=k₀₂V₂). In this model, V₁ and V₂ represent plasma volume and the ISF distribution volume as seen by the subcutaneously inserted sensor, respectively. To estimate the gradient and delay, the mass balance relationship for the ISF pool may be expressed as:

$$\frac{d {C}_{2}}{d t} = - ( {k}_{02} + {k}_{12} ) {C}_{2} + {k}_{12} \frac{{V}_{1}}{{V}_{2}} {C}_{1}$$

where C₁ and C₂ are the plasma and ISF glucose concentrations, respectively. The ISF-to-plasma glucose gradient and the ISF equilibration time constant (delay) are therefore determined according to:

$${C}_{1} = \frac{{k}_{12} + {k}_{02}}{{k}_{21} \frac{{V}_{1}}{{V}_{2}}} {C}_{2} ;$$

$$τ = \frac{1}{{k}_{12} + {k}_{02}}$$

(as described by K. Rebrin et al., Am J Physiol Endocrinol Metab 277:561-571, 1999)

The abovementioned modeling of plasma glucose from ISF glucose can be used to model, and thus determine, HbA1c levels from plasma glucose using a glucose sensor located in the subcutaneous tissue that measures glucose in the ISF.

In some embodiments, a model that directly derives the HbA1c value from sampled subcutaneous glucose measurements may be applied, such as the simple linear model described by Nathan D et al. (Diabetes Care 31(8), 1473-1478). For example, the regression equation for HbA1c and average glucose (AG) using a CGM is determined according to the relationship: AG=28*HbA1c-36.9.

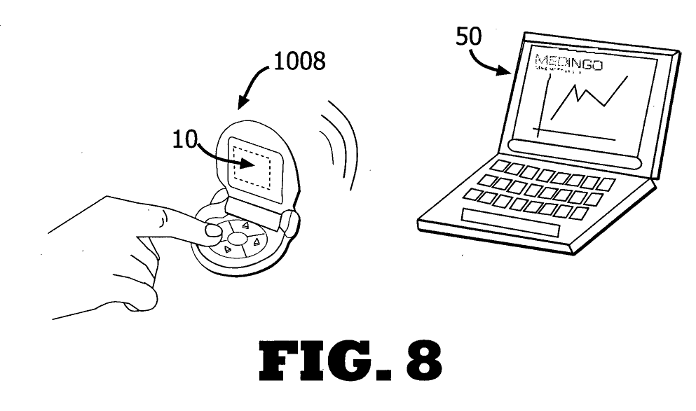

Referring to FIG. 8, a diagram of an exemplary embodiment of an HbA1c computation implementation using a remote control unit 1008 and an external PC 50 is shown. Determined HbA1c level values are stored and may be displayed in any graphical or non-graphical manner. In some embodiments, the saved data may automatically be sent (e.g., by electronic mail) to the patient's practitioner for evaluation.

Any and all patents, patent applications, articles and other published and non-published documents referred to anywhere in the subject disclosure are herein incorporated by reference in their entirety.

A number of embodiments of the invention have been described. Nevertheless, it will be understood that various modifications may be made without departing from the spirit and scope of the invention. Accordingly, other embodiments are within the scope of the following claims.

1.-25. (canceled)

26. A portable insulin dispensing system comprising:
a portable insulin dispensing pump to deliver insulin to a patient;
a glucose sensor to perform a plurality of periodic glucose measurements corresponding to glucose concentration in the blood; and
a processor to determine one or more HbA1c values representative of the patient's glycosylated hemoglobin levels based on the periodic glucose measurements.

27. The system of claim 26, wherein the processor is provided in any one or more of the portable insulin dispensing system, a handheld remote control unit for the insulin dispensing pump, and the glucose sensor.

28. The system of claim 26, wherein the glucose sensor is configured to perform a plurality of periodic glucose measurements in interstitial fluid.

29. The system of claim 26, wherein the glucose sensor is in communication with the dispensing pump.

30. The system of claim 26, wherein at least part of the pump is securable to the patient's skin.

31. The system of claim 26, wherein the pump comprises a disposable part and a reusable part.

32. The system of claim 26, wherein the one or more HbA1c values are determined based on at least one mathematical model relating the one or more HbA1c values to glucose concentrations determined from the glucose measurements.

33. The system of claim 32, wherein the at least one mathematical model comprises a first order reaction kinetics model representative of a first order reaction kinetics between glucose and hemoglobin and loss of HbA1c through erythrocyte clearance in which HbA1c is estimated to be a stable reaction product.

34. The system of claim 26, wherein the one or more HbA1c values are computed based on a time-weighted averaged computation of the glucose measurements.

35. The system of claim 28, wherein the one or more HbA1c values are determined based on at least one mathematical model relating the one or more HbA1c values to blood glucose concentrations, and wherein the blood glucose concentrations are determined based on at least one mathematical model relating the blood glucose concentrations to the glucose measurements in the ISF.

36. A method for monitoring glycemic control in a patient comprising:
delivering insulin into a body of the patient using an insulin dispensing device;
performing a plurality of periodic glucose measurements; and
determining one or more HbA1c values representative of the patient's glycosylated hemoglobin levels based on the periodic glucose measurements.

37. The method of claim 36, further comprising:
providing a processor in any one or more of the dispensing device, a handheld remote control unit for the insulin dispensing device and a glucose sensor, the processor configured to determine the one or more HbA1c values.

38. The method of claim 36, wherein performing the plurality of periodic glucose measurements comprises:
performing a plurality of glucose measurements in interstitial fluid.

39. The method of claims 36, wherein determining the one or more HbA1c values comprises:
computing the one or more HbA1c values based on at least one mathematical model relating the one or more HbA1c values to the glucose measurements.

40. The method of claim 39, wherein the at least one mathematical model comprises a first order reaction kinetics model representative of a first order reaction kinetics between glucose and hemoglobin and loss of HbA1c through erythrocyte clearance in which HbA1c is estimated to be a stable reaction product.

41. The method of claim 36, wherein computing the one or more HbA1c values comprises computing the one or more HbA1c values based on a time-weighted averaged computation of the glucose measurements.

42. The method of claim 36, wherein performing the plurality of periodic glucose measurements comprises performing periodic measurements of glucose concentration level of the patient using a continuous glucose monitoring unit (CGM).

43. The method of claim 38, wherein the one or more HbA1c values are determined based on at least one mathematical model relating the one or more HbA1c values to blood glucose concentrations, and wherein the blood glucose concentrations are determined based on at least one mathematical model relating the blood glucose concentrations to the glucose measurements in the ISF.

44. The method of claim 36, wherein performing the plurality of periodic glucose measurements comprises:
performing the plurality of periodic glucose measurements using a sensor in communication with the dispensing device.
